(19) United States
(12) Patent Application Publication
(10) Pub. No.: US 2020/0068935 A1
(43) Pub. Date: Mar. 5, 2020

(54) ESTRONETICS COMPOSITION

(71) Applicant: DuraScience Inc., New York, NY (US)

(72) Inventor: James Nitit Mah, New York, NY (US)

(73) Assignee: DuraScience Inc., New York, NY (US)

(21) Appl. No.: 16/679,280

(22) Filed: Nov. 10, 2019

(51) Int. Cl. A23L 33/105; A61K 36/232; A61K 36/481

(52) U.S. Cl. CPC A23L 33/105; A61K 36/481; A61K 36/232

(57) ABSTRACT

In an embodiment, a nutritional supplement composition is provided. The nutritional supplement composition is provided including a dong quai extract derived from the root of the angelica sinensis strain, astragalus extract from the astragalus membranaceus.strain and fenugreek extract from the trigonella foenum-graecum strain. The nutritional supplement is in the form of a compound known as ESTRONETICS™.

### BACKGROUND

In the past, we commonly used all parts of natural foods when we consumed a meal. Human eating patterns naturally accessed nutrients available from what was eaten and very little nutrients were wasted as a result. For example, when eating fish, the muscular parts of the fish were consumed along with other parts such as connective tissue. When eating plants, the edible portion of the plant was consumed in its entirety. Our bodies would then filter out what was not consumable, such as the outer shell of a kernel of corn for example. As this occurred, our bodies would extract what nutrients could be found in the food to sustain and grow the human form. This allowed use of local foods to provide nutrients which were available, but was somewhat limited in that foods not available locally could not be used in most cases. Over time, we learned to travel and to ship food from where it was produced to where it could be consumed. Additionally, we learned to prepare foods, removing portions of the food which were either inedible, or undesirable. Removing the inedible parts typically provided some benefit, while parts that were simply undesirable sometimes held valuable ingredients.

The angelica sinensis strain is a plant found in parts of Asia and used individually by people in China, Japan and Korea. It is a flowering plant with a fragrant smell. It is a member of the botanical family of carrots and celery.

The astragalus membranaceus.strain originates in China and parts of Asia. The astragalus membranaceus.strain is also known as huáng qi or milkvetch. The astragalus membranaceus.strain has been used in Chinese medicine for thousands of years.

The trigonella foenum-graecum strain is a strain commonly known as fenugreek. Fenugreek is an herb similar to clover that is native to the Mediterranean region, southern Europe, and western Asia. The seeds are used in cooking, to make medicine, or to hide the taste of other medicine. Fenugreek seeds smell and taste somewhat like maple syrup. Fenugreek leaves are eaten in India as a vegetable.

It is potentially useful to find a source of each of these items for use by consumers.

### SUMMARY OF THE INVENTION

A nutritional supplement composition is provided including a dong quai extract derived from the root of the angelica sinensis strain, astragalus extract from the astragalus membranaceus.strain and fenugreek extract from the trigonella foenum-graecum strain. The nutritional supplement is in the form of a compound known as ESTRONETICS™.

The foregoing, and other features and advantages of various embodiments of the invention, will be apparent from the following, more particular description of the embodiments of the invention, any accompanying drawings, and the claims.

### DETAILED DESCRIPTION

A composition is provided as ESTRONETICS™. The specific embodiments described in this document represent exemplary instances of the present invention, and are illustrative in nature rather than restrictive.

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments.

The composition (referred to as ESTRONETICS™) is preferably formed of (1) a dong quai extract derived from the root of the angelica sinensis strain, (2) astragalus extract from the astragalus membranaceus.strain and (3) fenugreek extract from the trigonella foenum-graecum strain.

The composition involves obtaining nutrients from a dong quai extract derived from the root of the angelica sinensis strain, extracting from the root a portion of the plant that provides a variety of potential benefits, for example. Dong quai is used to decrease menstrual and menopausal symptoms, such as cramps or premenstrual symptoms, and migraines, for example. Dong qual is also used as a blood purifier to manage or reduce hypertension and anemia. Moreover, dong quai is used to help with infertility, joint pain, ulcers and constipation, providing anti-inflammatory action in some instances, for example. Dong quai has also been used to treat skin issues related to loss of skin color (depigmentation) and psoriasis.

The composition further involves retrieving nutrients from an astragalus extract from the astragalus membranaceus.strain. This strain is known for providing a boost to the immune system and potentially aiding in management of heart disease among other qualities. The astragalus membranaceus.strain is a type of bean or legume and the extract comes from the bean or legume of the plant. Astragalus extract is understood to provide immune system boosts and reduction of free radicals along with associated damage to cells. Astragalus is also often used to assist with issues related to hay fever and other allergies, diabetes and kidney disease.

The composition also involves accessing nutrients from fenugreek extract from the trigonella foenum-graecum strain. The trigonella foenum-graecum strain originating in the Mediterranean, southern Europe and Northern Asia, is thought to provide benefits in a variety of areas. Fenugreek is used for kidney ailments, a vitamin deficiency disease called beriberi, mouth ulcers, boils, bronchitis, infection of the tissues beneath the surface of the skin (cellulitis), tuberculosis, chronic coughs, chapped lips, baldness, cancer, Parkinson's disease, and exercise performance. Fenugreek is taken by mouth for digestive problems such as loss of appetite, upset stomach, constipation, inflammation of the stomach (gastritis). Fenugreek is also used for diabetes, painful menstruation, menopause, polycystic ovary syndrome, arthritis, poor thyroid function, and obesity. It is also used for conditions that affect heart health such as “hardening of the arteries” (atherosclerosis) and for high blood levels of certain fats including cholesterol and triglycerides. Some men use fenugreek for hernia, erectile dysfunction (ED), male infertility, and other male problems. Both men and women use fenugreek to improve sexual interest. Women who are breast-feeding sometimes use fenugreek to promote milk flow.

### Results From Use of ESTRONETICS™

The composition includes a combination of the three extracts subjected to mixing and binding processes to further link the material provided therein. Typically, the combination of the extracts provides for a mix of building blocks for general healing and injury repair. The combination further provides specific materials having potential to aid in healing of specific types of injuries, enhancement of structures common in the skin and other parts of the body, and contribution to various aspects of general nutrition.

### Menopausal and Mentrual Moderation

Fenugreek has been found to alleviate menopausal and menstrual effects and symptoms, such as pre-menstrual symptoms, migraines, cramps and other similar effects. Additionally, dong quai is also seen to provide some similar effects in terms of moderating the effects of the menstrual cycle. Collectively, the ingredients, dong quai and fenugreek, have similar effects and can be expected to operate in concert, with varying effects depending on personal factors.

### Diabetes Control

The trigonella foenum-graecum strain has shown some effects which can assist in reducing the effects of diabetes. Such activity can depend on individual factors. Additionally, the angelica sinensis strain has also been shown to potentially have some diabetes control effects. These materials operate as part of the ESTRONETICS™ composition and thereby provide further benefits in those who struggle with glucose.

### Additional Beneficial Effects

Beyond the effects specific to control of diabetes and weight loss activity of the compound, other beneficial effects typically manifest. Reduction of inflammation resulting from presence of the dong quai extract can provide a variety of benefits. Moreover, the ingredients in combination can potentially provide for improved digestion and related improvements resulting from the combination of weight loss effect, diabetes moderating and reduced inflammation.

The benefit of the composition includes restoring and enhancing cells. Our cells constantly deteriorate in daily life. If there is no support to restore the deterioration, cellular damage will affect the body functions. ESTRONETICS™ composition is a supplementary factor that can restore the cells to the body balance without drug and substance usage. When cells are restored, in the next process, ESTRONETICS™ composition may accordingly boost cell strength, equivalent to increasing the effectiveness and the number of cells, while maximizing physical and mental abilities After the cellular restoration and boosting the cell strength, the next challenge is the protection to maintain the long-lasting quality of the cells. ESTRONETICS™ dietary supplement may help to protect and delay the cellular deterioration. This will also result in improving the immune system, as the immune system no longer needs to activate against inflammation or other forms of deterioration. When the cell functions are systematically restored, boosted and protected, ESTRONETICS™ dietary supplement may directly enhance physical and mental capacity to go beyond limits at each age and to live life fully as the pace of life requires a strong response to maintain youth and vigor.

The health benefits of ESTRONETICS™ are also potentially of value to children, and help to develop the strength of new cells and protect cells from early deterioration. Both are beneficial to the development of body, intelligence, memory, and positive emotions. The physical results may include i) restore cells for normal growth in each stage, ii) recover from illness, iii) build muscle, v) boost the immune system, vi) reduce the risk of the incidence of disease, vii) promote growth to maximum effectiveness, viii) adjust height, ix) improve body growth, x) efficient immunity and xi) increase energy. The wound and injury healing properties are potentially of particular assistance to children, as the nutrients provided assist specifically with structures often injured in sports or other childhood activities.

The composition also helps to restore the old damaged cells, repair damaged body tissues, delay the cellular deterioration, inhibit free radicals, and help the process of skin cell renewal in adults for glowing skin and younger appearance. Providing building blocks as previously mentioned contribute to enhancing balance, reducing exhaustion from stress and demands of healing, improving concentration and work efficiency, reducing healing time and longer term pain, and boosting the immune system to stay healthy

Physical results may also include: a radiant and vibrant skin, younger appearance; reducing fatigue at work; restoring the reproductive system; refreshing the body; boosting the immune system; protecting against free radical formation; and preventing premature aging.

The compounds are typically dried and ground or milled to small particles, and subsequently combined in the desired ratio. The combination may remain as a powder to be added to food or pressed in a tablet. Alternatively, the compounds may be mixed in an aqueous or other liquid solution and thereby provided in liquid form.

In an embodiment, a preferred dosage, the total compound blended is 400 mg, pressed into a single tablet. The compound may be sold under the ESTRONETICS™ name. The dosage may include a combination of 200 mg of dong quai extract derived from the root of the angelica sinensis strain, 50 mg of astragalus extract from the astragalus membranaceus strain and 150 mg fenugreek extract from the trigonella foenum-graecum strain. However, other ratios of the ingredients may be provided, and smaller or larger dosages may be used for particular applications.

### Study of ESTRONETICS™ Effectiveness

A study of users of a trial formulation was conducted. The study focused initially on dermatological factors, but also considered other possible results. Results were broken down into dermatological factors and other potentially related factors. Participants reported the following results:

Ninety percent (90%) of participants reported reduced increased balancing of estrogen levels, with a typical response of a seventy percent (90%) better estrogen balance as a measured result.

Ninety-four percent (94%) of participants reported decreased vaginal excretion during menstruation.

Ninety-five percent (95%) of participants reported reduced pain during intercourse due to better vaginal moisture levels. The participants reported pain decreasing to one tenth of incidence prior to use of ESTRONETICS™.

Ninety-five percent (95%) of participants reported better intravaginal immunity. The participants reported incidence of itch and undesired odor decreasing to one tenth of incidence prior to use of ESTRONETICS™.

Ninety-five percent (95%) of participants also reported improved firmness of breast tissue.

Ninety-two percent (92%) of participants reported better sleep quality.

Ninety-four percent (94%) of participants were found to have a ninety percent (90%) improvement in gut excretion and reabsorption of estrogen.

Ninety percent (90%) of participants reported reduced night sweating. The participants reported a ninety percent (90%) reduction in night sweating prior to use of ESTRONETICS™.

Eight-seven percent (87%) of participants reported improved emotional control after use of ESTRONETICS™.

Additionally, ninety percent (90%) of participants reported increased libido, typically nearly doubling their perceived libido after use of ESTRONETICS™.

These results came out of trial uses and a combination of user reporting and measurement of results for users of a trial formulation in a controlled study. Results may vary. However, the generally positive responses are expected to indicate a likelihood that many users of ESTRONETICS™ would experience some or all of these results to varying degrees depending on individual factors.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claim.

What is claimed is:

1. A nutritional composition consisting of:
a dong quai extract derived from the root of the angelica sinensis strain;
astragalus extract from the astragalus membranaceus strain; and
fenugreek extract from the trigonella foenum-graecum strain.

2. The composition of claim 1, wherein:
the dong quai extract is in the amount of 200 mg,
the astragalus extract is in the amount of 50 mg, and
the fenugreek extract is in the amount of 150 mg.
